(19) United States
(12) Patent Application Publication
(10) Pub. No.: US 2008/0262501 A1
(43) Pub. Date: Oct. 23, 2008

(54) Multi-function corpectomy instrument

(76) Inventors: Richard Evan Chen, Memphis, TN (US); William D. Armstrong, Memphis, TN (US); Zafar Saleem Khan, Newport Coast, CA (US)

Correspondence Address: KRIEG DEVAULT LLP, ONE INDIANA SQUARE, SUITE 2800, INDIANAPOLIS, IN, 46204-2709, US

(21) Appl. No.: 11/788,541

(22) Filed: Apr. 20, 2007

(51) Int. Cl. A61B 17/58; A61B 17/70; A61F 5/00; A61B 17/04

(52) U.S. Cl. 606/90; 606/86 A; 606/102; 606/301

(57) ABSTRACT

A multi-functional surgical instrument assembly for installing an implant between adjacent bone structures is disclosed that is capable of distracting adjacent bone structures, generating a measurement for a prosthetic implant, and installing the prosthetic implant without being removed from the patient. The surgical instrument includes a frame having a pair of opposing distracter arms movable toward or away from one another to a predetermined setting indicative of a height requirement of the implant. A pair of opposing spreader support segments is connected with a respective distal end of the distracter arms and extend downwardly a predetermined distance in relation to the distracter arms. A pair of opposing implant guide segments is connected with a distal end of the opposing spreader support segments. An inserter is detachably connected with the prosthetic implant, wherein the prosthetic implant is positioned between the opposing implant guide segments using the inserter.

### TECHNICAL FIELD

The present invention relates generally to the field of surgical instrumentation and methods, and more particularly relates to instrumentation and methods for distracting bone structures, measuring the height of a cavity formed between the adjacent bone structures, and inserting an implant with a surgical instrument in the cavity between the adjacent bone structures.

### BACKGROUND

In the treatment of diseases, injuries, or malformations affecting spinal motion segments, and especially those affecting the disc tissue, it has long been known to remove some or all of a degenerated, ruptured, or otherwise failing disc. In cases involving intervertebral disc tissue that has been removed or is otherwise absent from a spinal motion segment, corrective measures are taken to ensure the proper spacing of the vertebrae formerly separated by the removed disc tissue. Prosthetic implant devices may be inserted into the disc space to maintain the structural integrity of the spinal column.

Currently, a method does not exist to easily distract, measure, and insert an implant such as a vertebral body replacement device or spacer without having to remove the initial distraction. Existing alternative measurement methods include cutting surgical cotton swabs down to size or molding surgical wax into the desired shape after the initial distraction is removed. After the cotton swabs are cut down to size or the surgical wax is molded into the desired shape, a measurement of the cotton swabs or surgical wax is taken. Once the length or width of the implant needed is determined, the appropriately sized implant is implanted between the vertebral bodies. As such, a need exists for an instrument that will allow a surgeon to distract, read a measurement, and assemble and insert an implant device without removing the initial distraction.

### SUMMARY

A surgical instrument assembly for installing an implant between adjacent bone structures is disclosed that is capable of distracting adjacent bone structures, generating a height measurement associated with the required implant without removing the initial distraction, and implanting the implant into place between adjacent bone structures. The surgical instrument assembly includes a frame having a pair of opposing distracter arms that are movable toward or away from each other to a predetermined distraction setting indicative of a height requirement of the implant. A pair of opposing spreader support segments are connected with a respective distal end of the distracter arms and extend downwardly a predetermined distance in relation to the distracter arms. A pair of opposing implant guide segments is connected with a distal end of the opposing spreader support segments. An inserter is detachably connected with the prosthetic implant for positioning the implant between the opposing implant guide segments.

One or more of the distracter arms of the frame may be connected with the frame on a movable housing. The moveable housings allow the distracter arms to be positioned to a desired height setting, thereby distracting adjacent bone structures when positioned between the adjacent bone structures. The frame may also include a measurement display that is operable to generate a measurement indicative of the height or width that an implant should be in order to properly fit within a window formed between the adjacent bone structures. The measurement display may comprise markings on an upper surface of the frame that are spaced apart in predetermined distance increments (e.g.—millimeters). In order to determine the height measurement, the surgeon merely needs to look at the measurement display.

Another aspect of the present invention discloses a method of inserting a prosthetic implant between adjacent bone structures. A surgical instrument having a frame including opposing implant guide segments is inserted in a surgical window formed in the adjacent bone structures. The bone structures are then distracted to a predetermined distracted position using the surgical instrument. A height measurement associated with the prosthetic implant is then determined by the surgeon using a measurement generated from a measurement display on the frame. A height adjusted prosthetic implant may then be created having a height determined as a function of the height measurement. Once the prosthetic implant is formed having the proper height, it is inserted between opposing implant guide segments of the surgical instrument. The height adjusted prosthetic implant is then guided into place between the adjacent bone structures using the opposing implant guide segments.

Yet another aspect of the present invention discloses a system for inserting prosthetic implants between adjacent bone structures, such as adjacent vertebrae of the spine. The system includes a frame having a distracter segment including opposing guide segments moveable between an adjustable distracted position and a closed position. The opposing guide segments are operable to distract adjacent bone structures to a predetermined position. A prosthetic implant measurement display is included on the frame for generating a height measurement associated with an implant to be placed between adjacent bone structures as a function of the predetermined position. A height adjustable prosthetic implant is constructed to a height as a function of the height measurement. The height adjustable prosthetic implant is slidably engaged by the opposing guide segments to guide the height adjustable prosthetic implant between the adjacent bone structures.

Other systems, methods, features and advantages of the invention will be, or will become apparent to one with skill in the art upon examination of the following figures and detailed description. It is intended that all such additional systems, methods, features and advantages be included within this description, be within the scope of the invention, and be protected by the following claims.

### BRIEF DESCRIPTION OF THE DRAWINGS

The components in the figures are not necessarily to scale, emphasis instead being placed upon illustrating the principles of the invention. Moreover, in the figures, like reference numerals designate corresponding parts throughout the different views.

FIG. 1 illustrates a representative spinal column.

FIG. 2 is a cross-sectional view of a representative vertebra and associated vertebral disc of the spinal column.

FIG. 3 illustrates various planes in relation to a human body.

FIG. 4 illustrates a segment of a spinal column showing two adjacent vertebrae and the space or window produced after removal of an intervertebral disc.

FIG. 5 is a perspective view of a representative surgical instrument holding an implant prior to insertion between respective adjacent vertebrae.

FIG. 6 is a top view of the surgical instrument illustrated in FIG. 5.

FIG. 7 is a perspective view of a portion of the surgical instrument illustrated in FIG. 5.

FIG. 8 illustrates a cross-sectional view of a portion of the surgical instrument with an implant loaded in the surgical implant prior to insertion between adjacent bone structures.

FIG. 9 illustrates a cross-sectional view of a portion of the surgical instrument as an implant is being positioned in the window between the vertebrae.

FIG. 10 illustrates a cross-sectional view of a portion of the surgical instrument as it is removed from the vertebrae while leaving the implant in place.

### DETAILED DESCRIPTION

A multi-functional surgical instrument 200 is disclosed that is used to install implantable devices, including prosthesis suitable for implantation within the body to restore and/or augment connective tissue such as intervertebral discs. In various embodiments, the implantable devices comprise devices designed to replace missing, removed or resected body parts or structure. The implantable devices, apparatus or mechanisms are configured such that the devices can be formed from parts, elements or components which alone, or in combination, comprise the implantable device. The implantable devices can also be configured such that one or more elements or components are formed integrally to achieve a desired physiological, operational or functional result such that the components complete the device. Functional results can include the surgical restoration and functional power of a joint, controlling, limiting or altering the functional power of a joint, and/or eliminating the functional power of a joint by preventing joint motion. Portions of the implantable devices can be configured to replace or augment existing anatomy and/or implanted devices, and/or be used in combination with resection or removal of existing anatomical structure.

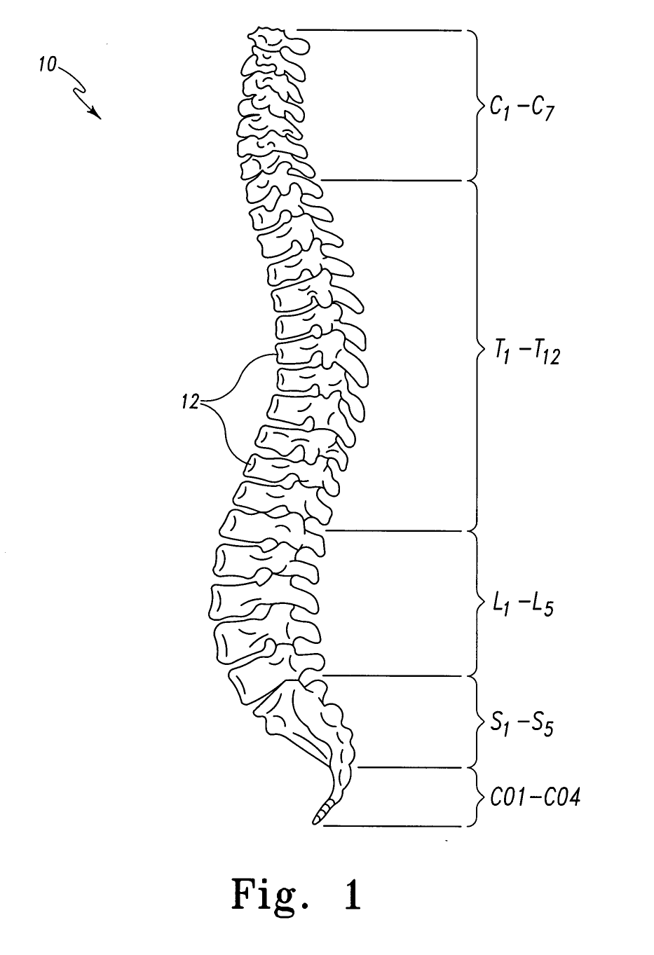

The implantable devices disclosed herein are preferably, but not exclusively, designed to interact with the human spinal column 10, as shown in FIG. 1, which is comprised of a series of thirty-three stacked vertebrae 12 divided into five regions. The cervical region includes seven vertebrae, known as C1-C7. The thoracic region includes twelve vertebrae, known as T1-T12. The lumbar region contains five vertebrae, known as L1-L5. The sacral region is comprised of five fused vertebrae, known as S1-S5, while the coccygeal region contains four fused vertebrae, known as Co1-Co4. Although the preferred embodiment is designed to interact with the human spinal column 10, the present invention could also be taken advantage of in conjunction with any implantable device that may be inserted or placed near nerve tissue.

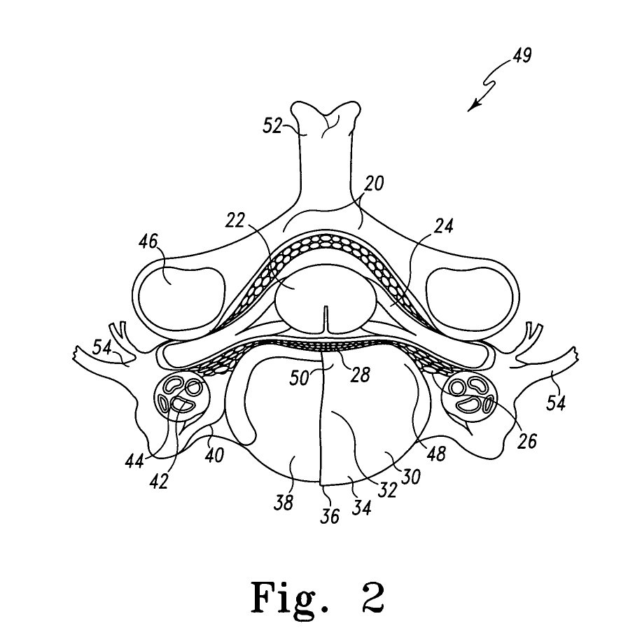

FIG. 2 illustrates a cross-sectional anatomical view of a respective vertebra 49 and associated intervertebral disc 30. Structures of a typical cervical vertebra 49 (superior aspect) are shown in FIG. 2 and include a lamina 20, a spinal cord 22, a dorsal root of spinal nerve 24, a ventral root of spinal nerve 26, a posterior longitudinal ligament 28, a intervertebral disc 30, a nucleus pulposus 32, a annulus fibrosus 34, a anterior longitudinal ligament 36, a vertebral body 38, a pedicle 40, a vertebral artery 42, vertebral veins 44, a superior articular facet 46, a posterior lateral portion of the annulus 48, a posterior medial portion of the annulus 50, a spinous process 52, and a spinal nerve 54. In FIG. 2, one side of the intervertebral disc 30 is not shown so that the anterior vertebral body 38 can be seen.

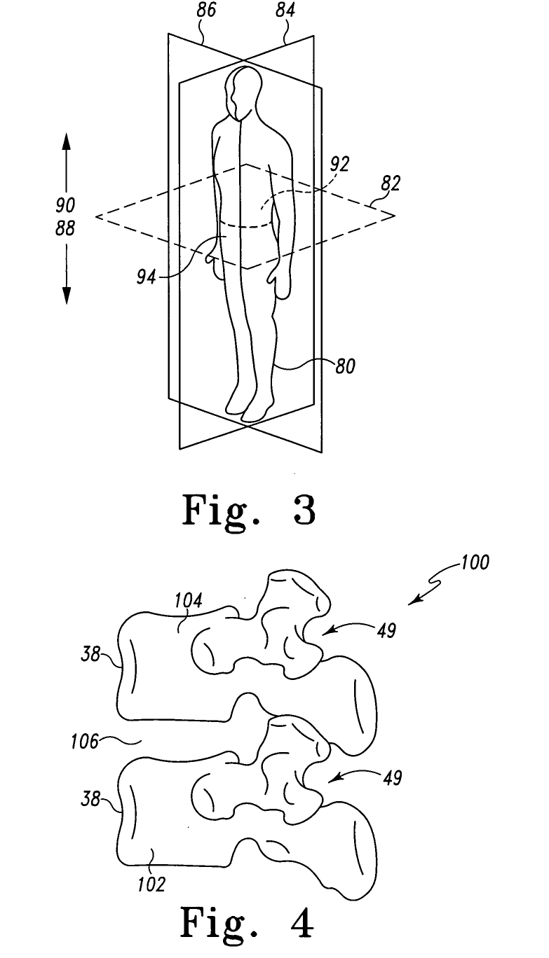

Referring to FIG. 3, in order to understand the configurability, adaptability, and operational aspects of the invention, it is helpful to understand the anatomical references of the body 80 with respect to which the position and operation of the devices, and components thereof, are described. There are three anatomical planes generally used in anatomy to describe the human body and structure within the human body: the axial plane 82, the sagittal plane 84 and the coronal plane 86. Additionally, devices and the operation of devices are better understood with respect to the caudal 88 direction and/or the cephalad direction 90. Devices positioned within the body 80 can be positioned dorsally 92 (or posteriorly) such that the placement or operation of the device is toward the back or rear of the body. Alternatively, devices can be positioned ventrally 94 (or anteriorly) such that the placement or operation of the device is toward the front of the body. Various embodiments of the surgical instrument and systems of the present invention may be configurable and variable with respect to a single anatomical plane or with respect to two or more anatomical planes. In addition, the devices disclosed herein may be inserted in the body 80 using a posterior approach, an anterior approach, a lateral approach or a combination thereof.

Referring to FIG. 4, for further example, two vertebrae 49 will be discussed, designated as spinal segment 100 including a lower vertebrae 102 and an upper vertebrae 104 each having a vertebral body 38. In one embodiment, some or the entire intervertebral disc 30 that would have been positioned between the two vertebrae 102, 104 is removed via a discectomy or a similar other procedure. Removal of the diseased or degenerated disc 30 results in the formation of an intervertebral cavity or window 106 between the lower and upper vertebrae 102, 104. This window 106 is where prosthetic discs or implants 110 are inserted when some or all of a respective intervertebral disc 30 is removed.

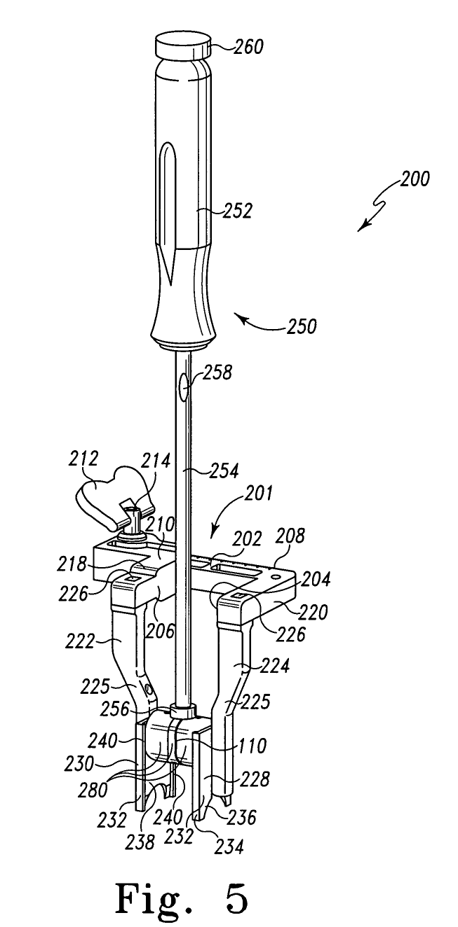

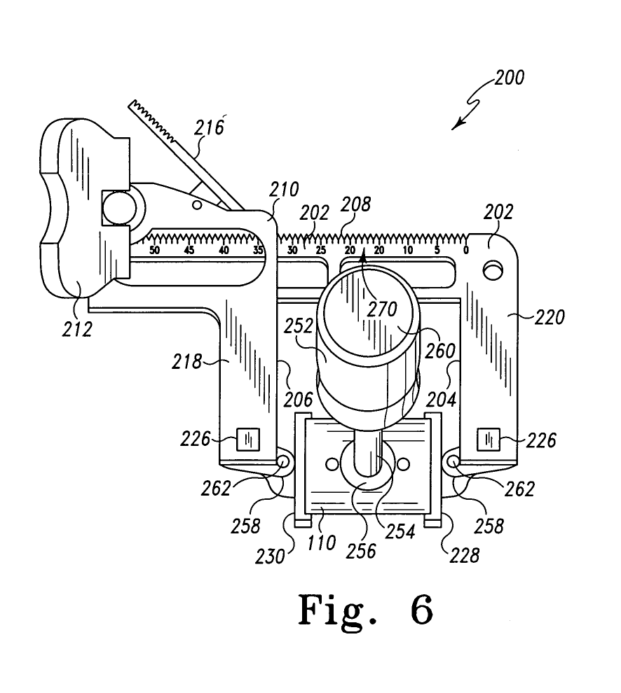

Referring to FIGS. 5 and 6, a multi-functional surgical instrument 200 is set forth that is operable to distract respective adjacent vertebrae 49 (See FIG. 4) to create a surgical window 106, to measure a height associated with the surgical window 106, and to insert a prosthetic implant 110. The surgical instrument 200 is capable of being placed through an incision into the surgical window 106 and spreading to distract vertebral bodies 38 associated with adjacent vertebrae 49 for viewing and access to the surgical window 106 formed by the removal of some or all of a respective intervertebral disc 30. Once placed in a distracted or spread open position, components of the surgical instrument 200 may be used to obtain a measurement of the proper height a prosthetic disc 110 should be for insertion into the window 106. After the prosthetic disc 110 is properly sized as a function of the height measurement obtained from the surgical instrument 200, components of the surgical instrument 200 are used to guide the prosthetic implant 110 into place within the window 106 between the adjacent vertebrae 49.

The surgical instrument 200 includes a frame 201 that is comprised of a crossbar 202 with opposing ends 204, 206, which define right and left distracter arms, respectively. A longitudinal side edge of the crossbar 202 includes a rack gear 208. As set forth in greater detail below with respect to the left distracter arm 206, although the right distracter arm 204 is illustrated as being an integral part of the crossbar 202, it should be recognized that the right distracter arm 204 may also be removably and movably connected with the crossbar 202 in a ratchet or sliding manner as well. The left distracter arm 206 is mounted to the crossbar 202 on a movable housing 210, having a foldable crank handle 212 for rotating a pinion gear 214 engaging the rack gear 208. Rotation of the crank handle 212 operates the pinion gear 214 and moves the left distracter arm 206 towards or away from the right distracter arm 204. A plurality of frames 201 may be used for multi-level procedures.

The movable housing 210 includes a spring loaded releasable arm lock 216 that secures the movable distracter arm 206 in place to the crossbar 202 after the movable distracter arm 206 has been properly positioned. The releasable arm lock 216 engages the rack gear 208 to secure the moveable housing 210 in place. The releasable arm lock 216 keeps the movable distracter arm 206 from moving unless the releasable arm lock 216 is pressed toward the rack gear 208. Optionally, the rack gear 208 may be designed to allow the pinion gear 214 to further spread open the surgical instrument 200 in a ratcheting manner without releasing the releasable arm lock 216 from the rack gear 208. If the releasable arm lock 216 is pressed toward the rack gear 208, the pinion gear 214 may be used to move the movable distracter arm 206 up and down the rack gear 208. Once the releasable arm lock 216 is released, because it is spring loaded, it engages the rack gear 208 thereby preventing further movement of the movable distracter arm 206.

The movable distracter arm 206 includes a first outwardly protruding member 218 that extends a predetermined distance in a generally horizontal direction away from a proximate end of the crossbar 202. The fixed distracter arm 204 includes a second outwardly protruding member 220 that extends a predetermined distance in substantially the same direction as the outwardly protruding member 218 of the movable distracter arm 206. At the distal end of the first outwardly protruding member 218 is a spreader support segment 222 that extends downwardly a predetermined distance from the distal end of the first outwardly protruding member 218. A second spreader support segment 224 extends downwardly from the distal end of the second outwardly protruding member 220 in substantially the same direction as the first spreader support segment 224. In one embodiment, the distal ends of the first and second outwardly protruding members 218, 220 include an aperture 226 that allows the spreader support segments 222, 224 to be removably connected with the distal ends of the first and second outwardly protruding members 218, 220.

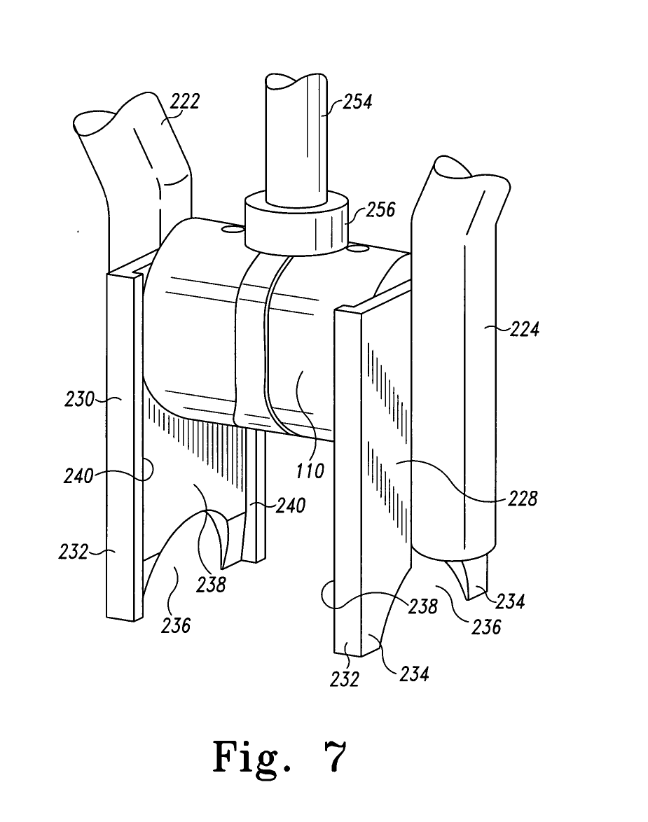

Referring to FIGS. 5 and 7, at a distal end of the spreader support segments 222, 224 are a pair of opposing implant guide segments 228, 230. The implant guide segments 228, 230 are used to guide the prosthetic implant 110 into the window 106 between adjacent vertebrae 49. The implant guide segments 228, 230 include a tip portion 232 located at a distal end of the implant guide segments 228, 230. The tip portion 232 of the implant guide segments 228, 230 may include a tapered portion 234 that assists in the insertion of the implant guide segments 228, 230 between adjacent vertebrae 49. In addition, the distal end of the tip portion 232 of the implant guide segments 228, 230 may include a cutout portion 236 that further assists the insertion and distraction process during a surgical procedure. The distal end of the tip portion 232 may be viewed as a blade portion or segment of the tip portion 232. The implant guide segments 228, 230 may be removably connected with the spreader support segments 222, 224 to allow for use of guide segments 228, 230 that fit implants of various footprints.

The implant guide segments 228, 230 include a recessed track portion or segment 238 located at a proximal end of the implant guide segments 228, 230 operable to selectively contain the prosthetic implant 110 such that the prosthetic implant 110 is guided in a loading direction in relation to the adjacent vertebrae 49. The term loading direction should be broadly construed to include any direction in which, as set forth in greater detail below, the prosthetic implant 110 may be inserted between adjacent bone structures, which are illustrated as adjacent vertebrae 49 in this representative embodiment. As illustrated, each recessed track portion 238 includes opposing side walls 240 that form a track or guide for the prosthetic implant 110 to fit and slide within.

Referring collectively to FIGS. 5-7, the surgical instrument 200 also includes an inserter 250. In one aspect, the inserter 250 includes a handle portion 252 that is connected with a shaft portion 254. A connector portion 256 of the inserter 250 is located at a distal end of the shaft portion 254. The connector portion 256 of the inserter 250 is operable to be detachably connected with a side surface of the prosthetic implant 110. Although not specifically illustrated, the prosthetic implant 110 may include a pair of pin holes that allow pins of the connector portion 246 to detachably connect with the prosthetic implant 110. A detachment release mechanism 258 is included on the shaft portion 254 of the inserter 250 to detach the prosthetic implant 110 from the inserter 250 once properly placed in position in the window 106 between adjacent vertebrae 49. The connector portion 246 and the prosthetic implant 110 may also include mating threaded portions in alternative embodiments that may be used to detachably interconnect the two respective elements. Various other methods of detachably connecting the connector portion 246 of the inserter 240 with the prosthetic implant 110 are envisioned as being capable of being used in conjunction with the present invention (e.g.—friction fit and biocompatible temporary adhesives to name a few).

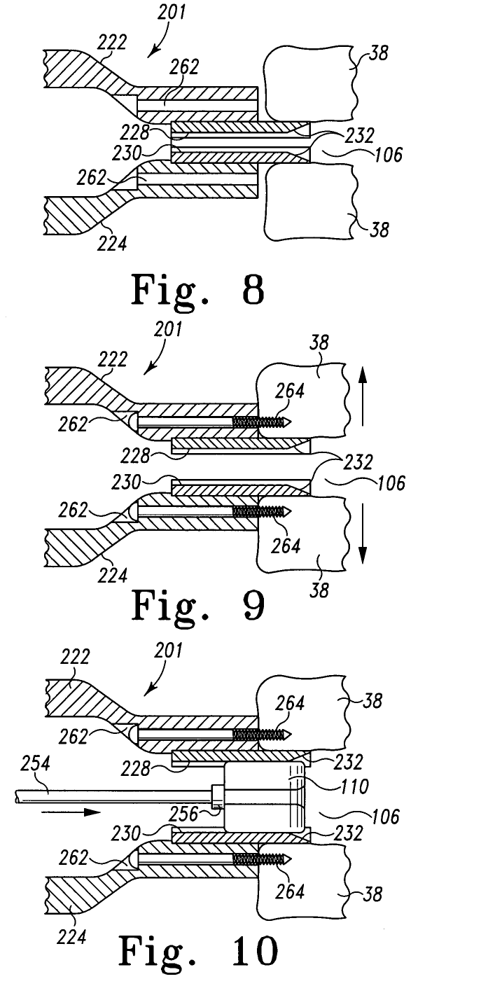

Referring back to FIG. 5, the spreader support segments 222, 224 may curve inwardly towards one another at a predetermined point 225 in one embodiment of the present invention. As illustrated best in FIG. 6, an upper surface 258 created by the inward curve of the spreader support segments 222, 224 may include a screw aperture 262 that runs through the entire length of the spreader support segments 222, 224. Referring to FIGS. 9 and 10, the screw apertures 262 may be used to secure the frame 201 to the vertebral bodies 30 of the adjacent vertebrae 49. As depicted, two surgical bone screws 264 may be used to temporarily secure the frame 201 to the adjacent vertebrae 49. During the surgical procedure, the surgeon places the bone screws 264 through the screw apertures 262 and then screws the bone screws 264 into the vertebral bodies 38 of the vertebrae 49. Although not specifically illustrated, the spreader support segments 222, 224 could be flipped in order to allow for both a left handed and right handed surgical approach.

Referring to FIG. 8, during a surgical procedure the tip portions 234 of the implant guide segments 228, 230 of the frame 201 are placed between adjacent respective vertebrae 49. The implant guide segments 228, 230 may be inserted between the adjacent vertebrae 49 to a depth approximately equivalent to the width of the prosthetic implant 110. For example, this may be 11 mm for 14 mm wide rails, 14 mm for 16 mm wide rails, and 16 mm for 18 mm wide rails. However, the depth inserted into the window 106 between the vertebral bodies 38 could be anything as long as it allows for proper distraction of the vertebral bodies 38. If the surgeon uses the bone screws 264 to secure the frame 201 to the vertebral bodies 38, a small portion of the tip portion 234 of the implant guide segments 228, 230 should be sufficient. Casper pins may be used instead of bone screws.

Referring to FIG. 9, once in position a predetermined depth in the window 106 of the adjacent vertebrae 49, in one embodiment, the frame 201 may be secured to the vertebral bodies 38 by placing bone screws 264 into the bone screw apertures 262 and screwing the bone screws 264 into the vertebral bodies 38. However, use of the bone screws 264 to secure the frame 201 to the vertebral bodies 38 should be viewed as optional. Once the bone screws 264 are securely in place, the frame 201 is expanded to distract the adjacent vertebrae 49 a predetermined distance apart from one another to assist in the reception of the implant 110. As previously set forth, the foldable crank handle 212 is rotated by the surgeon to thereby rotate a pinion gear 214 that engages the rack gear 208. As the pinion gear 214 is rotated a predetermined direction, the implant guide members 228, 230 spread apart from one another thereby distracting the vertebral bodies 38 from one another.

Referring to FIG. 10, once the frame 201 is securely in place, the implant 110 is inserted between the respective implant guide segments 228, 230 and may then be tapped into place in the window 106 between the vertebral bodies 38 by using a hammer or the like. A metallic cap 260 (See FIG. 5) may be connected with the top of the inserter 250 so that the surgeon may hammer or tap the implant 110 in place. Once the implant 110 is tapped into proper alignment or positioning, the frame 201 may be released, thereby allowing the vertebrae 49 to retract or return to their normal position with the implant lying between the adjacent vertebral bodies 38. The inserter 250 may then be detached from the implant 110 and placed out of the way. Once the inserter 250 is clear of the surgical area or out of the way, the surgeon may remove the frame 201 from the vertebral bodies 38. If bone screws 264 are used to secure the frame 201 to the vertebral bodies 38, the bone screws 264 must first be removed before the frame 201 can be removed from the surgical site.

Referring back to FIG. 6, the frame 201 of the surgical instrument 200 includes measurement markings or a measurement indicator 270 on an upper surface of the crossbar 202. Once the tip portions 232 of the implant guide segments 228, 230 have been inserted in the window 106 and distracted to a predetermined state, the surgeon may use the measurement indicator 270 on the frame 201 to determine the proper height of the prosthetic implant 110 to be implanted. In this embodiment, the measurement of the required implant 110 is determined by the surgeon by looking at the measurement indicator 270 when the frame 201 is in a distracted state. The measurement indicator 270, which in this embodiment comprises 1 millimeter incrementally spaced markings on an upper surface of the frame 201, provides the surgeon with a visual indication of the distance in which the vertebrae 49 have been distracted, thereby allowing the surgeon to determine the proper size of the implant 110. The scale of the measurement indicator 270 can be modified if other increments are deemed preferable.

The measurement indicator 270 may be placed such that an inside edge of the fixed distractor arm 204 starts at zero and a second inside edge of the movable housing 210 is used to provide the measurement indication. In other embodiments, the measurement indicator 270 may be offset a predetermined distance from the inside edge of the fixed retractor arm 204 to compensate for the fact that the spreader support segments 222, 224 curve inwardly toward one another to allow room for the screw apertures 262 that are used to anchor the frame 201 to the vertebral bodies 38. It should be appreciated that several other placement arrangements may be utilized in other embodiments of the present invention.

Once the measurement is obtained, the prosthetic implant 110 may then to constructed, to the proper height, and then inserted between the vertebral bodies 38 of the adjacent vertebrae 49. Some prosthetic implants 110 may come in a variety of sizes thereby allowing the surgeon to select an appropriately sized implant. As such, the surgeon is capable of determining the height of the prosthetic implant 110 without the necessity of removing the surgical instrument 200 or the distraction created by the surgical instrument 200. As such, the surgical instrument is operable to distract vertebral bodies 38, generate and display a measurement of a proper height associated with a prosthetic implant 110, and insert the height adjusted prosthetic implant 110 between the adjacent vertebral bodies 38 without ever having to remove the surgical implant 200.

Referring back to FIG. 5, the prosthetic implant 110 may comprise a plurality of interconnected stacked discs 280. The interconnected stacked discs 280 may come in a variety of shapes and sizes depending on the particular application in which the prosthetic implant 110 is utilized. Those skilled in the art should recognize that the thickness or width of the prosthetic implant 110, and thereby the height of the prosthetic implant 110 in the illustrated embodiment, is adjusted by stacking different sized discs 280 on top of one another to arrive at the desired height. The desired height corresponds to the approximate width of the recessed tracks 238 in the opposing implant guide segments 228, 230.

Although the preferred embodiment of the present invention is disclosed for use with vertebral bodies, those skilled in the art should recognize that the disclosed surgical instrument 200 may have application in other areas of the body as well. In addition, although FIGS. 8-10 illustrate the present invention being utilized to expand two adjacent vertebral bodies so that an implant replacing removed disc tissue may be inserted, it should be appreciated that the present invention may also be used in surgical procedures that involve either partial or complete removal of a vertebral body. Referring to FIG. 1, as an example, the superior endplate could be touching C4 and the inferior endplate could be touching C6 such that an implant may be inserted where C5 would otherwise be located if it had not been removed. As such, the surgical instrument 200 may be used to replace disc tissue and/or vertebral bodies.

While the invention has been illustrated and described in detail in the drawings and foregoing description, the same is to be considered as illustrative and not restrictive in character.

What is claimed is:

1. A surgical instrument assembly for installing an implant between adjacent bone structures, comprising:
a frame having a pair of opposing distracter arms movable toward or away from one another to a predetermined distraction setting, wherein said frame includes a measurement indicator operable to generate a measurement display associated with a correct size of said implant;
a pair of opposing spreader support segments connected with a respective distal end of said distracter arms and extending downwardly a predetermined distance in relation to said opposing distracter arms;
a pair of opposing implant guide segments connected with a distal end of said opposing spreader support segments; and
an inserter detachably connected with said implant, wherein said implant is positioned between said opposing implant guide segments of said frame using said inserter.

2. The surgical instrument assembly of claim 1, wherein a first opposing distracter arm is positioned on a movable housing connected with said frame.

3. The surgical instrument assembly of claim 2, wherein said movable housing includes a releasable arm lock for locking said first opposing distracter arm in place to said frame.

4. The surgical instrument assembly of claim 2, wherein said movable housing includes a pinion gear engaged with a gear track located on said frame.

5. The surgical instrument assembly of claim 4, wherein a crank handle is connected with said pinion gear thereby allowing a surgeon to turn said pinion gear to move said movable housing up or down said gear track.

6. The surgical instrument assembly of claim 1, wherein said opposing spreader support segments move inwardly toward one another a second predetermined distance as said opposing spreader support segments extend downwardly said predetermined distance thereby forming an upper surface on each respective spreader support segment.

7. The surgical instrument assembly of claim 6, wherein said upper surface includes an aperture running through each said respective opposing spreader support segment for receiving a bone screw to secure said frame to a respective one of said adjacent bone structures.

8. The surgical instrument assembly of claim 1, wherein said frame includes a measurement display operable to generate a height measurement associated with said implant.

9. The surgical instrument assembly of claim 1, wherein said opposing implant guide segments include a recessed track portion.

10. The surgical instrument assembly of claim 9, wherein said recessed track portion is formed by two opposing side walls of each respective opposing implant guide segment.

11. The surgical instrument assembly of claim 10, wherein said implant moveably fits between said recessed track portions of said opposing implant guide segments.

12. The surgical instrument assembly of claim 1, wherein said inserter comprises a handle segment, a shaft segment, and a connector segment.

13. A method of inserting a prosthetic implant between adjacent bone structures, comprising:
inserting a surgical instrument having a frame including opposing implant guide segments in a surgical window formed in said adjacent bone structures;
distracting said bone structures to a predetermined distracted position using said surgical instrument;
determining a height measurement associated with said prosthetic implant using a measurement generated from a measurement display on said frame;
forming a height adjusted prosthetic implant having a height determined as a function of said height measurement;
inserting said height adjusted prosthetic implant between said opposing implant guide segments of said surgical instrument; and
guiding said height adjusted prosthetic implant between said adjacent bone structures using said opposing implant guide segments.

14. The method of claim 13, wherein said opposing implant guide segments include a recessed track segment that guides said height adjusted prosthetic implant between said adjacent bone segments.

15. The method of claim 14, wherein said recessed track segment includes opposing side walls that secure a predetermined portion of upper and lower surfaces of said implant slidably engaged in said opposing implant guide segments.

16. The method of claim 13, further comprising connecting said height adjusted prosthetic implant with an inserter.

17. The method of claim 16, further comprising using said inserter to insert said height adjusted prosthetic implant between said opposing implant guide segments.

18. The method of claim 13, further comprising removing said surgical instrument from said adjacent bone structure leaving said height adjusted prosthetic implant between said adjacent bone structures.

19. The method of claim 13, further comprising securing said surgical instrument to said adjacent bone structures.

20. The method of claim 19, wherein said surgical instrument is secured to said adjacent bone structures with one or more bone screws.

21. The method of claims 13, further comprising tapping an inserter connected with said height adjusted prosthetic implant to position said height adjusted prosthetic implant between said adjacent bone structures.

22. A system for inserting prosthetic implants between bone structures, comprising:
a frame including a distracter segment having opposing guide segments moveable between an adjustable distracted position and a closed position, wherein said opposing guide segments are operable to distract adjacent bone structures to a predetermined position;
a prosthetic implant measurement display for generating a height measurement associated with an implant to be placed between adjacent bone structures as a function of said predetermined position; and
a height adjustable prosthetic implant constructed to a height as a function of said height measurement, wherein said height adjustable prosthetic implant is slidably engaged by said opposing guide segments to guide said height adjustable prosthetic implant between said adjacent bone structures.

23. The system of claim 22, wherein said distracter segment includes a pair of opposing distracter arms connected with a crossbar.

24. The system of claim 23, wherein a pair of opposing spreader support segments is connected with a distal end of said distracter arms.

25. The system of claim 24, wherein a respective opposing guide segment is connected with a respective opposing spreader segment.

26. The system of claim 25, wherein said opposing guide segments include a recessed track segment.

27. The system of claim 25, wherein said opposing guide segments include a tapered tip portion.

28. The system of claim 22, wherein said opposing guide segments are connected with respective opposing spreader support segments, wherein said spreader support segments include a bone screw aperture for securing said frame to said adjacent bone structures.

29. A method, comprising:
inserting a distracter between adjacent bone structures;
distracting said adjacent bone structure to a predetermined distracted position; and
generating a height display associated with a prosthetic implant to be inserted between said adjacent bone structure with said distracter;
forming said prosthetic implant as a function of said height display;
guiding said prosthetic implant between said adjacent bone structures; and
removing said distracter from said adjacent bone structures.

30. The method of claim 29, wherein said prosthetic implant is guided between said adjacent bone structures by placing an upper and lower surface of said prosthetic implant between a pair of opposing implant guide segments on said distracter that are inserted between said adjacent vertebrae.

31. The method of claim 29, wherein said height display is generated on a numerical measurement display extending on an upper surface of said distracter.

32. The method of claim 29, further comprising anchoring said distracter in said adjacent bone structures using one or more bone screws.
